(19) United States
(12) Patent Application Publication
(10) Pub. No.: US 2026/0108192 A1
(43) Pub. Date: Apr. 23, 2026

(54) URINE AND OTHER LIQUID SAMPLE TEST SYSTEMS AND METHODS

(71) Applicant: Regina Ehlman, Denver, CO (US)

(72) Inventor: Regina Ehlman, Denver, CO (US)

(21) Appl. No.: 19/362,144

(22) Filed: Oct. 17, 2025

Related U.S. Application Data
(60) Provisional application No. 63/709,484, filed on Oct. 20, 2024.

(51) Int. Cl. A61B 5/20; A61B 5/145; A61B 10/00; A61B 50/30

(52) U.S. Cl. CPC A61B 5/207; A61B 5/14507; A61B 50/30; A61B 10/007; A61B 2050/314; A61B 2560/0431; A61B 2562/0271; A61B 2562/0295; A61B 2562/12

(57) ABSTRACT

A test system for urine or other liquid samples includes a package initially formed to be flat and planar, and in a sealed or closed state to thereby define an interior cavity of the package. The liquid sample test system includes at least one test reagent for testing a liquid sample to be contained within the interior cavity of the package. The at least one test reagent is contained within the interior package in the sealed or closed state. The package is capable of being opened by a user on a top side of the package to expose the interior cavity of the package to an exterior of the package to thereby enable access to the at least one test reagent through an opening of the package in an opened state, and placement of the liquid sample into the package through the opening of the package.

### CROSS-REFERENCE TO RELATED APPLICATIONS

This application claims the benefit of and priority to U.S. Provisional Patent Application No. 63/709,484 filed on Oct. 20, 2024, which is incorporated by reference herein in its entirety.

### TECHNICAL FIELD

The present disclosure is generally directed toward testing of liquids and, in particular, toward devices useful for both collecting and testing samples of a liquid under test.

### BACKGROUND

Testing a urine sample is a common technique used in both home and clinical settings for useful purposes such as checking for pregnancy or ovulation hormones and checking for the presence of alcohol or drug substances and their metabolites. FIGS. 1A-1C depict a known system 1 (or kit) and method for performing, for example, a urine test to determine if pregnancy hormones are present in a human being. Testing urine samples for analytes other than hormones associated with human pregnancy may utilize a similar approach. As shown in FIGS. 1A and 1B, the known system 1 may include a urine test strip 15 provided in an interior cavity 10 of a rectangular-shaped package 5 that may be sealed over its four sides. A means 20 (e.g., cuts, perforations, or the like) for facilitating opening a first side 25 of the package 5 opposite a second side 30 of the package 5 may be present.

A user (not shown in FIGS. 1A-1C) of the known system 1 may grasp a corner section 35 of package 5 and tear the first side 25 of package 5 off to thereby expose the interior cavity, as shown in FIG. 1C. Having done so, the test strip 15 is made accessible for use in a urine test of the subject. The test strip 15 may be made of an absorbent material and have a first end 50 and a second end 60 opposite the first end 50. The first end 50 of test strip 15 may be positioned in interior cavity 10 proximate to the first end 25 of the package 5. As such, the user may grasp the first end 50 of test strip 15 to then transfer it from package 5 to a cup 45 the user (or patient) has deposited a suitable volume of urine 40 in, as shown by the arrow 65 in FIG. 1C. A section or portion 55 of test strip 15 proximate to the second end 60 thereof provides the functional area for reaction with hormones, drugs, and any other analyte(s) present in the urine 40 sample, and will not typically come into contact with a user's fingers during use. As such, the user places the second end 60 of test strip 15 into the urine 40 in the cup 45 for a specified period of time and may then read the result (e.g., a change of color and/or the appearance of one or more colored lines) of the test (e.g., pregnant vs. not pregnant).

While the individual package 5 may lie flat and thus be densely packed with additional packages 5 to make efficient use of storage space, this is not so for cups 45. Use of the cup 45 as part of system 1 may be desirable for users to urinate in given that its opening 70 can be larger and thereby provide a larger target than an opening 80 provided by the exposed cavity 10 after removal of the first end 25 of package 5. However, that manner of use of system 1 necessitates a greater amount of storage space, and also increases both costs of materials and associated waste. In some cases, the user of the known system 1 may attempt to urinate into the so opened package 5 or directly onto the portion 55 of the test strip 15 having the functional area.

Given the smaller target involved and greater manual dexterity required, this latter manner of using system 1 is typically more messy and unpleasant as compared to urinating directly into the cup 45. Furthermore, in that latter use case of known system 1, the greater difficulty involved may necessitate repeating a test or have a higher risk of inaccurate results. Even if a user does successfully urinate a sufficient volume into an open package 5, the flat second side 30 (one of the two short sides of the rectangular-shaped package 5) of the opened package 5 having urine 40 may not stand upright as can cup 45. As such, it is difficult to place, wait a prescribed period of time and then later remove, the test strip 15 from the urine 40 contained in package 5 in order to successfully complete a urine sample test. Such problematic aspects of the known system 1 and others like it may lead its users to be discouraged from taking proper care to perform the test correctly due to their frustration. Similar challenges may arise for known systems and methods for testing liquids other than urine.

Thus, a urine and other liquid sample testing system and associated methods solving the aforementioned problems and providing additional advantages is desired.

### SUMMARY

A first aspect of the present disclosure provides a test system for liquid samples. The test system may include a test strip for testing a liquid sample. The system may also include a flat and planar package. The package may be initially formed in a sealed or closed state. In that initial state, the package may define an interior cavity to contain the test strip therein. The package may have at least three sides. The package may be configured to be opened on a top side of the at least three sides of the package to expose the interior cavity to thereby enable access to the test strip by a user of the test system. The package in an opened state may have an opening at its top.

In some embodiments of the test system according to the first aspect of the disclosure, the top side of the package may have a length that is greater than at least one other side of the at least three sides of the package. In one embodiment, the package may include a fold up/pop out structure positioned proximate to a bottom of the package and coupled to at least a portion of interior surfaces of the interior cavity to thereby enable the bottom of the package to be expanded outward. In an example, the fold up/pop out structure positioned proximate to the bottom of the package and coupled to the at least a portion of interior surfaces of the interior cavity may enable, with the bottom of the package in an expanded state, maintenance of the package in the opened state with the opening at the top of the package remaining open. In some embodiments, maintenance of the package in the opened state with the opening at the top of the package remaining open further enables dispensing or placement by the user of the liquid sample into the exposed interior cavity through the opening at the top of the package. In another embodiment, the liquid sample may be, or may include, urine and the maintenance of the package in the opened state with the opening at the top of the package remaining open may enable urination by the user into the exposed interior cavity through the opening at the top of the package. In yet another embodiment, the fold up/pop out structure positioned proximate to the bottom of the package and coupled to the at least a portion of interior surfaces of the interior cavity may enable, with the bottom of the package in an expanded state, stable placement of the bottom of the package on a surface to thereby enable the package in the opened state to stand upright on the surface.

In one embodiment of the test system according to the first aspect of the disclosure, the flat and planar package is rectangle shaped having two long sides and two short sides, and wherein a top side of the package is a first side of the two long sides. In another embodiment, the package is trapezoid shaped having at least a first side and a second, shorter side parallel to, and opposite, the first side. In yet another embodiment, the package is square shaped, where a first side of the package is a top side thereof positioned opposite a second side of the package. In still another embodiment, the package is triangle shaped, and wherein a first side of the package is a top side thereof.

In some embodiments, the top side of the package may include means for opening the package via removal of at least part of the first side to thereby expose the interior cavity and create the opening at the top of the package. In one embodiment, the liquid sample may be, or may include urine, and the test strip may be capable of qualitatively or quantitatively determining a presence or level of one or more analytes in the liquid sample. In that case of urine, for example, the test strip may be capable of qualitatively or quantitatively determining at least one of: a pregnancy status, an ovulation status, and a level or presence of at least one of: one or more drugs, one or more drug metabolites, one or more proteins, glucose, one or more biomarkers, and ketone. In other embodiments, the test strip may be capable of testing for: a presence or level of chlorination in, or a pH of, the liquid sample. In an example, the test system may include two or more test strips contained within the interior cavity of the package in the sealed or closed state. In an example, each test strip of the two or more test strips is for testing a different analyte in the liquid sample. In another example, at least two test strips of the two or more test strips are for testing the same analyte in the liquid sample.

In one embodiment, the test system may include a thermometer positioned sufficiently with respect to the interior cavity of the package to enable a user to visually determine a temperature of the liquid sample contained in the package in the opened state. In an example, the thermometer is a strip thermometer coupled to an interior or exterior surface of the package such that a level of a liquid under test will be at or above a position of the strip thermometer. In another embodiment, the test system may include a handle. In some embodiments, the test system may include means for alternately sealing and unsealing the package in the opened state.

A second aspect of the present disclosure provides a liquid sample test system. The liquid sample test system may include a package initially formed to be flat and planar, and in a sealed or closed state to thereby define an interior cavity of the package. The liquid sample test system may include at least one test reagent for testing a liquid sample to be contained within the interior cavity of the package, wherein the at least one test reagent is contained within the interior package in the sealed or closed state. The package may be capable of being opened by a user on a top side of the package to expose the interior cavity of the package to an exterior of the package to thereby enable: access to the at least one test reagent through an opening of the package in an opened state; and placement of the liquid sample into the package through the opening of the package.

In some embodiments liquid sample test system of the second aspect of the disclosure, the at least one test reagent may be, or may include, a material in the form of at least one of a liquid and a solid. The at least one test reagent may be capable of qualitatively or quantitatively determining a presence or level of one or more analytes in the liquid sample. In an example, the at least one test reagent may be, or may include, at least one test strip. In an example, the test reagent(s) may be present inside of an unopened package of the test system in a sub-package of its own that a user of the test system would open before, during, or after a liquid sample under test is dispensed into or otherwise provided to an opened package. In another example, the test reagent(s) may be present inside of the unopened package attached or otherwise affixed or coupled to an interior surface of the package. In that case, the reagent(s) (e.g., test strip) may come into contact with a liquid sample under test provided into the opened package of the test system.

In yet another embodiment, the test reagent(s) are present in the unopened package without any subpackaging or being affixed to an interior surface of the package. In the case of a solid or liquid test reagent, the user may provide the liquid into the opened package and if the reagent(s) react with an analyte of interest in the sample, either the reagent(s) or the liquid sample under test may undergo a color change, for example, that indicates either the present or the absence of the analyte of interest. For a test strip in this case, a user may be able to view a color change indicating the presence or absence of an analyte of interest without removing the strip if the liquid sample under test is sufficiently clear. In another case of a solid or liquid test reagent, the user may first remove the reagent(s) from the opened package, then provide the liquid sample under test into the opened package, and then place the reagent(s) into the liquid sample. An example of this latter case is a test strip being provided to a liquid sample after the sample is placed into the opened package.

A third aspect of the present disclosure provides a kit for testing a liquid sample. The kit may include a test system according to, for example and without limitation, any of the embodiments according to the first and/or the second aspects of the disclosure. The kit may include instructions for a user to follow for testing a liquid sample using the test system. In one embodiment, the instructions of the kit may be printed on paper. In an example, the instructions of the kit may be contained inside of the interior cavity of the package in the sealed or closed state along with the test reagent(s). In another example, the instructions of the kit may be attached to a portion of an exterior surface of the package. In some embodiments, the instructions of the kit for using the test system may be printed on at least a portion of the exterior surface of the package.

A fourth aspect of the present disclosure provides a method of manufacturing a test system for liquid samples. The method may be applied to, for example and without limitation, manufacturing any of the embodiments summarized above with reference to the first, second, and third aspects of the disclosure. The method may include the step of providing a package for the test system in an at least partially unfinished state. The method may include the step of providing at least one test reagent for the test system. The method may include the step of finishing the package with the at least one test reagent positioned in an interior cavity of the package in a finished state.

In some embodiments of the method of the fourth aspect of the disclosure, the method may include the step of forming at least one means for easing or otherwise facilitating the opening of one side of the package in its finished state. In an example, forming the at least one means for easing or otherwise facilitating the opening may be implemented, at least in part, in the method while the package is in the at least partially unfinished state.

In one embodiment, the method may include the step of forming means for alternately sealing and unsealing the package in an opened state during use. In an example, forming the means for alternately sealing and unsealing may be implemented, at least in part, in the method while the package is in the at least partially unfinished state.

In another embodiment, the method may include forming a fold up/pop out structure

positioned proximate to a bottom of the package and coupled to at least a portion of interior surfaces of the interior cavity to thereby enable the bottom of the package to be expanded outward. In an example, forming the fold up/pop out structure may be implemented, at least in part, in the method while the package is in the at least partially unfinished state.

In yet another embodiment, the method may include the step of forming a handle configured to extend at or near normal from a side of the package during use thereof. In an example, forming the handle may be implemented, at least in part, in the method while the package is in the at least partially unfinished state. In an example, forming the handle may be implemented, at least in part, in the method while the package is in the finished state.

In still another embodiment, the method may include the step of positioning a thermometer sufficiently with respect to the interior cavity of the package to enable a user to visually determine a temperature of the liquid sample contained in the package in the opened state. In an example, positioning the thermometer may be implemented, at least in part, in the method while the package is in the at least partially unfinished state. In an example, positioning the thermometer may be implemented, at least in part, in the method while the package is in the finished state.

In some embodiments, the providing step of the method may include providing the package of the test system with a rectangle, square, trapezoid, or triangle shape. In another embodiment, the finishing step of the method may include the step of sealing all sides of the package to thereby place the package in the finished state.

### BRIEF DESCRIPTION OF THE DRAWINGS

One or more embodiments of the present invention are illustrated by way of example and not limitation in the figures of the accompanying drawings, in which like references indicate similar elements.

FIG. 1A is a plan view of a known system for performing a urine sample test.

FIG. 1B is a side view of the known system shown in FIG. 1A.

FIG. 1C is a perspective view of the known system of FIGS. 1A and 1B in an open top state and also depicts a known use case thereof.

FIG. 2A is a plan view of a system for performing a urine sample test, according to some embodiments of the present technology.

FIG. 2B is a side view of a long side of the system shown in FIG. 2A.

FIG. 2C is a side view of a short side of the system shown in FIG. 2A.

FIG. 2D is a perspective view of the system shown in FIGS. 2A-2C and also depicts a use case thereof.

FIG. 3A is a plan view of a system for performing a urine sample test, according to another embodiment of the present technology.

FIG. 3B is a perspective view of the system shown in FIG. 3A and also depicts a use case thereof.

FIG. 4A is a plan view of a system for performing a urine sample test, according to yet another embodiment of the present technology.

FIG. 4B is a side view of a long side of the system shown in FIG. 4A.

FIG. 4C is a side view of a short side of the system shown in FIG. 4A.

FIGS. 5A and 5B are pan views of example component parts of the system shown in FIGS. 4A-4C, according to some embodiments of the present technology.

FIG. 6A is a top view of the system shown in FIGS. 4A-4C in an open top and expanded state, and assembled or otherwise formed of the component parts shown in FIG. 5A, according to still other embodiments of the present technology.

FIG. 6B is a bottom view of the system shown in FIG. 6A.

FIG. 6C is a perspective view of the system shown in FIGS. 6A and 6B and also depicts a use case thereof.

FIG. 7A is a plan view of a system for performing a urine sample test, according to still another embodiment of the present technology.

FIG. 7B is a side view of a long side of the system shown in FIG. 7A.

FIG. 7C is a side view of a short side of the system shown in FIG. 7A.

FIG. 8 is a pan view of example component parts of the system shown in FIGS. 7A-7C, according to some embodiments of the present technology.

FIG. 9A is a top view of the system shown in FIGS. 7A-7C in an open top and expanded state, and assembled or otherwise formed of the component parts shown in FIG. 8, according to still other embodiments of the present technology.

FIG. 9B is a bottom view of the system shown in FIG. 9A.

FIG. 9C is a side view of the system shown in FIGS. 9A and 9B.

FIG. 9D is a perspective view of the system shown in FIGS. 9A-9C.

FIG. 9E is a side view of the system shown in FIG. 9D.

FIG. 10 is a flow chart of a method of manufacturing a test system for testing liquid samples according to some embodiments of the present technology.

### DETAILED DESCRIPTION

The following description and the appended drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

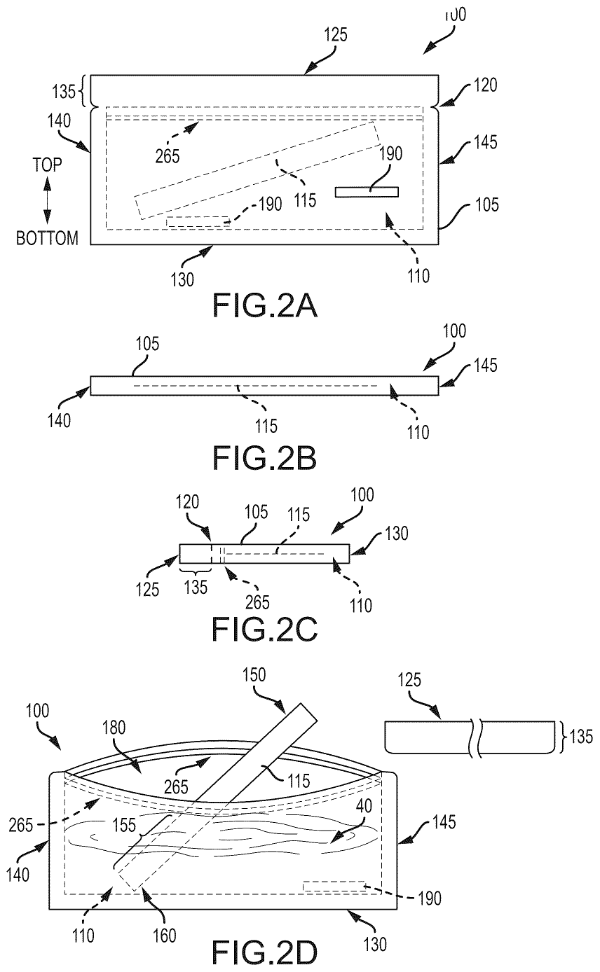

FIG. 2A is a plan view of a system 100 for performing a urine sample test, according to some embodiments of the present technology. FIG. 2B is a side view of a long side of the system 100 shown in FIG. 2A. FIG. 2C is a side view of a short side of the system 100 shown in FIG. 2A. FIG. 2D is a perspective view of the system 100 shown in FIGS. 2A-2C and also depicts a use case thereof.

Referring now to FIG. 2A, system 100 may include a package 105. Package 105 may be rectangular shaped having two long sides (125, 130), and two short sides (140, 145). At least one test reagent (e.g., a urine test strip 115) may be provided in an interior cavity 110 of the package 105. It can be appreciated by persons having ordinary skill in the art that the technology disclosed herein may be applied to testing liquid samples for a wide variety of analytes of interest. In a non-limiting example, the test strip 115 may be configured and/or otherwise capable of enabling a user of system 100 to determine if hormones associated with pregnancy are present in a urine sample of a human or other mammal. As will be appreciated, however, upon reading this disclosure, several other urine sample test analytes may be tested for using system 100 (e.g., drugs or their metabolites, protein, glucose, and ketones, among other possible analytes of interest). In an example, package 105 may be provided as two equally shaped and dimensioned rectangular package sides. Each such package side may be adhered, sealed, fused, or otherwise securely coupled to each other on their two short sides (140, 145) and one of their two long sides (e.g., second sides 130 thereof). Prior to adhering the second of the two long sides (e.g., first sides 125 thereof), test strip 115 may be placed into the interior cavity 110. Then, the first sides 125 of the aforementioned rectangular package sides of package 105 may be adhered, sealed, fused, or otherwise coupled to each other to thereby provide the final sealed package 105 of system 100. After that, the interior cavity 110 of the sealed package resulting from the above-described process may be airtight and also protected from exterior elements (e.g., moisture). In some examples, an expiration date of the urine test strip 115 as contained in interior cavity 110 may be indicated by printing or stamping on an outside surface of package 105.

Still referring to FIG. 2A, either before, concurrent with, or after the sealed package is made with test strip 115 contained within interior cavity 110, at least one means 120 (e.g., cuts, notches, perforations, or the like) for easing or otherwise facilitating opening the first, top, side 125 of the package 105 opposite the second side 130 of the package 5 may be formed on portion(s) of one or both of the two short sides (140, 145) proximate to the first long side 125. In the example illustrated in FIG. 2A, two such means 120 are shown as notches on both short sides (140, 145) of package 105, where such means 120 further define two (e.g., top) corner sections 135 of package 105. The above-described means 120 are configured to, or otherwise capable of, enabling a user of system 100 to open first side 125 of package 105 to thereby expose interior cavity 110 of the previously sealed package 105 and thereby gain access to it to remove the test strip 115 for use.

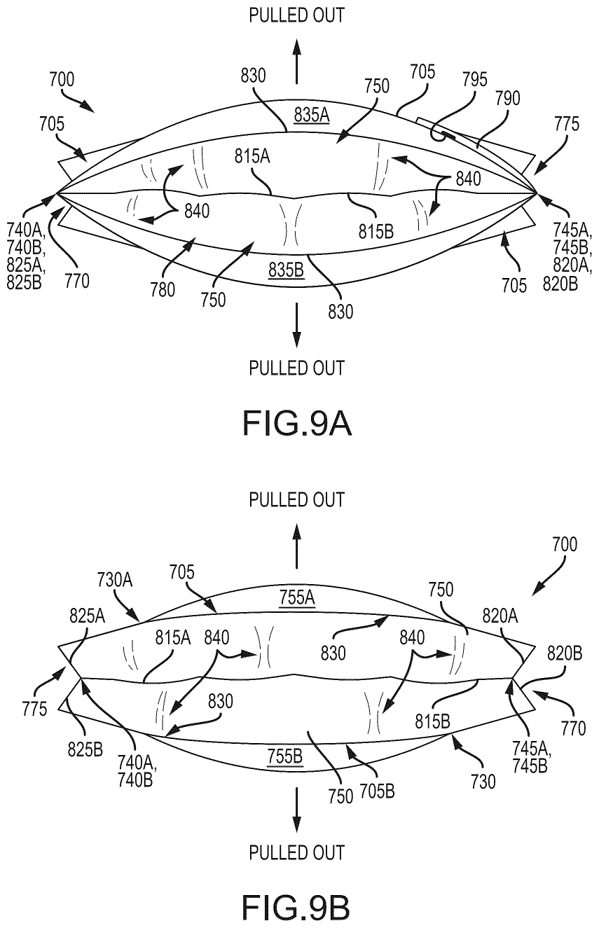

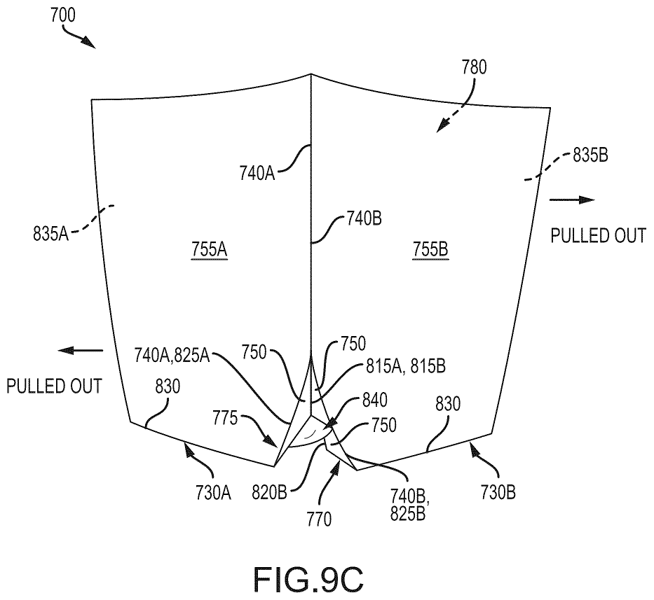

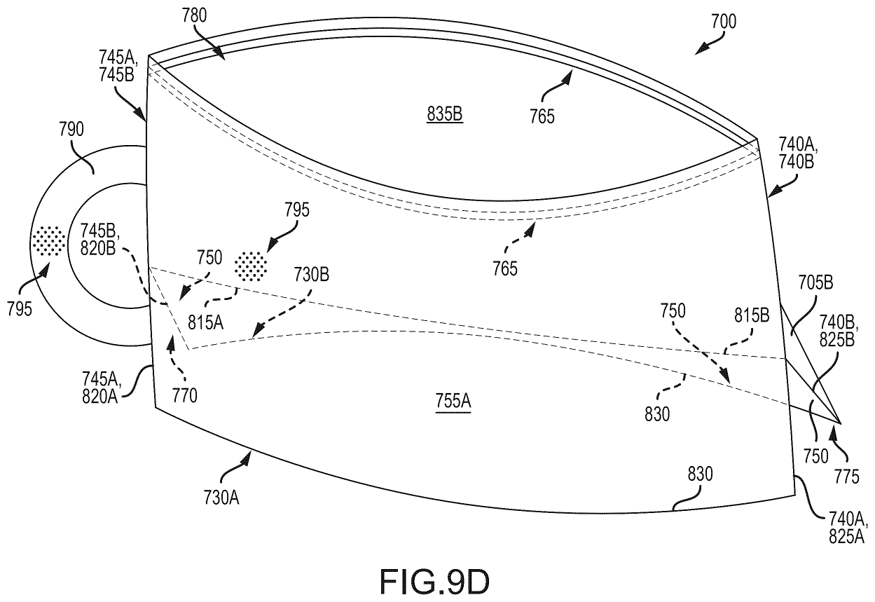

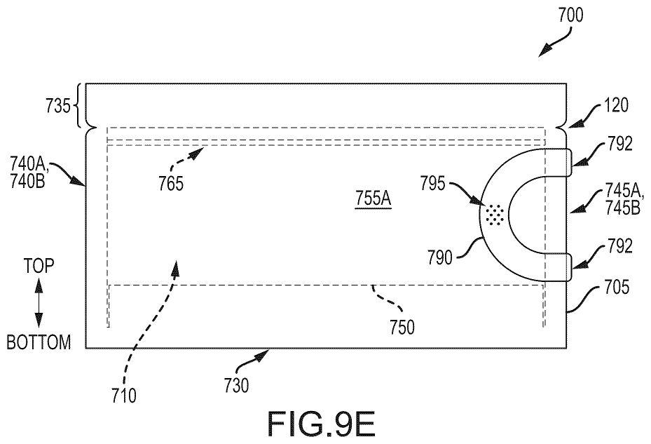

In some embodiments, the package 105 containing test strip 115 may be sterilized using heat, steam, irradiation, and any other suitable technique or combination of techniques. Sterilization of system 100 may be useful in applications where a sample of urine collected may, either instead of or in addition to, being tested for analyte(s) of interest using test strip, be cultured for microorganisms (e.g., bacteria, parasites, etc.) or viruses and/or subject to additional tests such as polymerase chain reaction (PCR) to determine a presence of an infection in a subject patient. In such examples, and also in general use, of system 100 may, as a part of, or in addition to, means 120, have a means 265 for alternately sealing and unsealing an opened package 105 of system 100, as shown in FIGS. 2A and 2D. In this way, package 105 may be resealable which may be beneficial in use cases such as a user desiring to move package 105 with liquid contained in it from place to place, or for storage of package 105 containing urine 40 or other liquid either before or after a test thereof. One example of means 265 is a press-and-seal closure (e.g., Ziploc) with a zipper track. A variant on that example is the slider-lock of pull-tab type of closure having a slider piece that is pulled back forth to alternately close and re-open a zipper track. In either case, the means 265 may have a single or a plurality (e.g., 2 or 3) of zipper tracks. Another example of means 265 is press-and-seal or press-to-close closures without zipper tracks. Such examples may use adhesive or cohesive materials, and rely on surface tackiness rather than interlocking tracks. Yet another example of means 265 is peel-and-stick or similar re-sealable adhesive strips that utilize pressure-sensitive adhesive (PSA). Referring to FIGS. 9D and 9E, some embodiments (e.g., liquid test system 700) similarly include means 765 for alternately sealing and unsealing an opened package (e.g., 705). It can be appreciated that the discussion of means 265 with reference to FIGS. 2A and 2D is equally applicable to means 765 for system 700 as described herein with reference to FIGS. 9D and 9E.

Referring now to FIGS. 2B and 2C, system 100 having urine test strip 115 contained in the interior cavity 110 of package 105 may lie substantially flat. As such, a plurality of individual system 100 packages 105 may be provided to users of system 100 in a suitably sized box or bag, for example, for convenient and space-efficient storage.

Referring now to FIG. 2D, a user (not shown in FIGS. 2A-2D) of system 100 may grasp the corner section 135 of package 105 and tear off or otherwise remove (e.g., by way of means 120) the first side 125 of package 105 to thereby expose the interior cavity 110. Having done so, the test strip 115 is made accessible for use in a test of the subject (e.g., the user or a patient of the user). The test strip 115 may be made of an absorbent material and have a first end 150 and second end 160 opposite the first end 150. In some embodiments, the first end 150 of the as packaged test strip 115 may be positioned in interior cavity 110 proximate to the first side 125 of the package 105, while the opposing second end 160 of test strip 115 of the as packaged test strip 115 may be positioned in interior cavity 110 proximate to the second side 130 of the package 105. A functional area 155 (e.g., that portion having reactive materials that would enable user to visualize a positive test result for analytes of interest present in urine 40) may be positioned on and/or in the material of test strip 115 proximate to the second end 160 thereof.

In an example use case, a user performing a test of a sample of human urine 40 may use one or two of his or her hands to squeeze short sides 140 and 145 of unsealed, opened package 105 together to create and further widen an opening 180 of the opened package 105. The test subject may then urinate a sample of urine 40 into interior cavity 110 through opening 180 while holding short sides 140 and 145 of package 105. In an example of the present use case, the user may next read his or her result directly from the functional area 155 of test strip 115 submersed in the urine 40 sample. In another example, the user (e.g., patient) providing the sample of urine 40 may display the submersed test strip 115 to another person (e.g., medical practitioner) to read the result displayed on their test strip 115 functional area 155.

In another example of a known use case, the urine sample test subject may be a mammal other than a human being. In such cases, the user of system 100 may conveniently position the opening 180 of the unsealed, opened package 105 to collect the urine sample from the non-human mammal test subject, and then proceed according to the above-described process for determining the test result for analyte(s) of interest. Accordingly, system 100 may find advantageous uses in both human and veterinary diagnostic applications in clinical settings, at home, or in the field. It can be appreciated from study of the present disclosure that system 100 can be used to test for analytes on any liquid sample of interest. System 100 is not limited just to urine sample testing of human or animal subjects. One such non-limiting example may be testing of pH or chlorination level of water samples from a swimming pool or hot tub.

In any of the above-described use case examples according to FIG. 2D, thermometer(s) 190, when present, may provide to the user of system 100 a visual indication of the temperature of the urine 40 sample contained in the interior cavity 110 of package 105. This may be particularly useful in a clinical setting in order to detect attempted subterfuge, as in the context of urine sample test for prohibited drug substances present in a human or animal subject. Should a urine sample of such a subject be indicated by thermometer(s) 190 to be outside an expected range of temperatures given a particular period of time elapsing since collection of the urine 40 sample, the sample may be flagged for further investigation (e.g., resampling of the test subject, etc.).

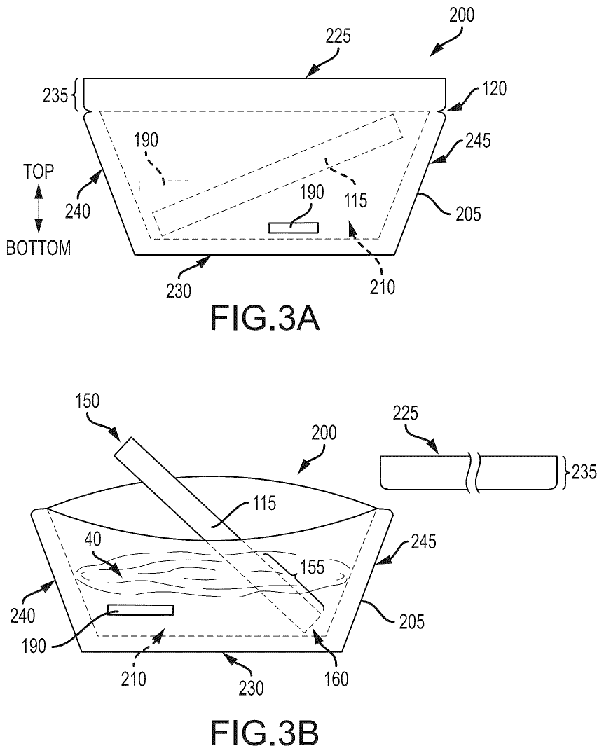

FIG. 3A is a plan view of a system 200 for performing a urine sample test, according to another embodiment of the present technology. FIG. 3B is a perspective view of the system 200 shown in FIG. 3A and also depicts a use case thereof. Side views of system 200 lying flat are substantially similar to those shown for system 100 in FIGS. 2B and 2C. In an analogous way as described above with reference to FIGS. 2A-2D, system 200 includes package 205, interior cavity 210, urine sample test strip 115, means 120 (e.g., cuts, notches, perforations, or the like) for user to open (e.g., unseal) the otherwise sealed package 205, corner sections 235, and thermometer(s) 190. As compared to system 100, package 205 of system 200 may have a trapezoidal shape. As such, package 205 may include a first side 225 and a shorter, second side 230 parallel to, and opposite, the first side 225.

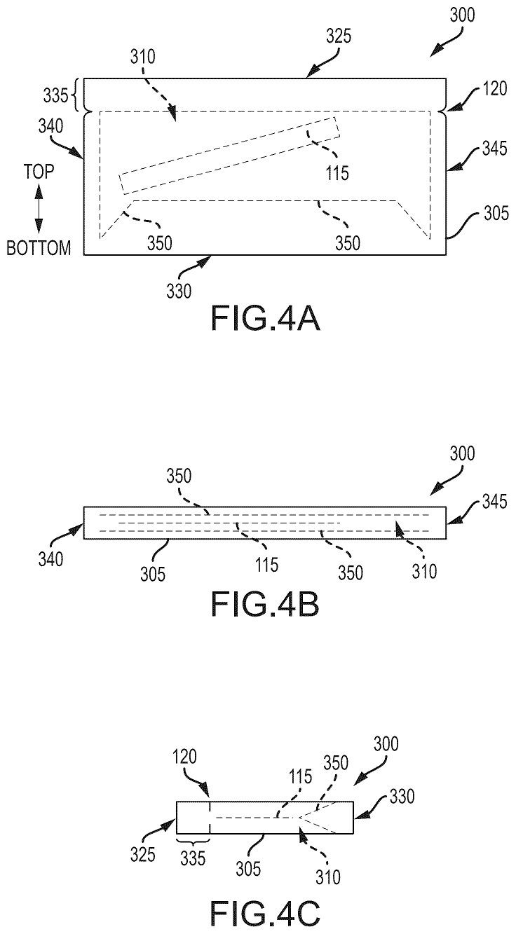

Package 205 may also include two sides (240, 245) connecting ends of the first 225 and the second side 230. The two sides (240, 245) may have equivalent lengths. In an example, each of the two sides (240, 245) are the same length as the second side 230. In another example, each of the two sides (240, 245) are shorter than the second side 230. In yet another example, each of the two sides (240, 245) are longer than the second side 230. In some embodiments, system 200 may include the aforementioned means 120 positioned proximate to the first side 225 and having the same, or analogous, structure and functionality as described above with reference to FIGS. 2A-2D. A sealed package 205 with test strip 115 contained within its interior cavity 210 for use in system 200 may be formed from two package sides in like manner as described above with reference to FIGS. 2A-2D. The use case illustrated in FIG. 3B follows an equivalent, or analogous, description as provided above with reference to FIG. 2D in all respects. In still other embodiments, the package of such a system according to the present technology as described herein with respect to FIGS. 2A-2D and 3A-3B (as well embodiments shown and described herein with reference to FIGS. 4A-4C, 5, and 6A-6D) may be of a triangular shape (e.g., equilateral, isosceles, or scalene). In yet other embodiments, the package of such a system according to the present technology as described herein with respect to FIGS. 2A-2D and 3A-3B (as well embodiments shown and described herein with reference to FIGS. 4A-4C, 5, and 6A-6D) may be of a square shape (e.g., equilateral, isosceles, or scalene). In such examples, not shown in the figures, the fold up/pop out structure 350 may be adjusted accordingly to provide the same, or analogous, advantageous ends as described above with respect to FIGS. 4A-4C, 5, and 6A-6C.

FIG. 4A is a plan view of a system 300 for performing a urine sample test, according to yet another embodiment of the present technology. FIG. 4B is a side view of a long side of the system 300 shown in FIG. 4A. FIG. 4C is a side view of a short side of the system 300 shown in FIG. 4A. In an analogous manner as described above with reference to FIGS. 2A-2D, system 300 includes package 305, interior cavity 310, urine sample test strip 115, means 120 (e.g., cuts, notches, perforations, or the like) for user to open (e.g., unseal) the otherwise sealed package 305, corner sections 335, first 325 and second sides 330, short sides (340, 345), and thermometer(s) 190.

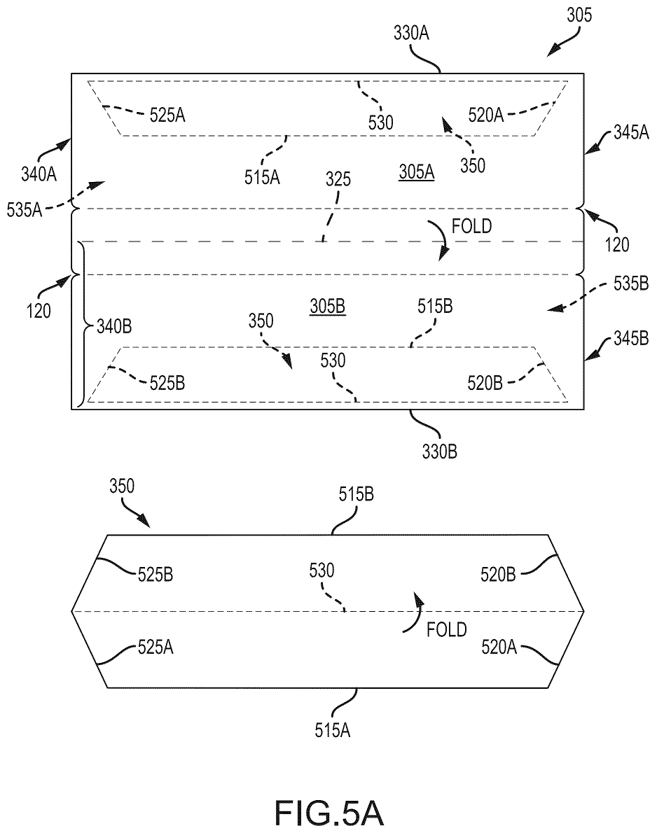

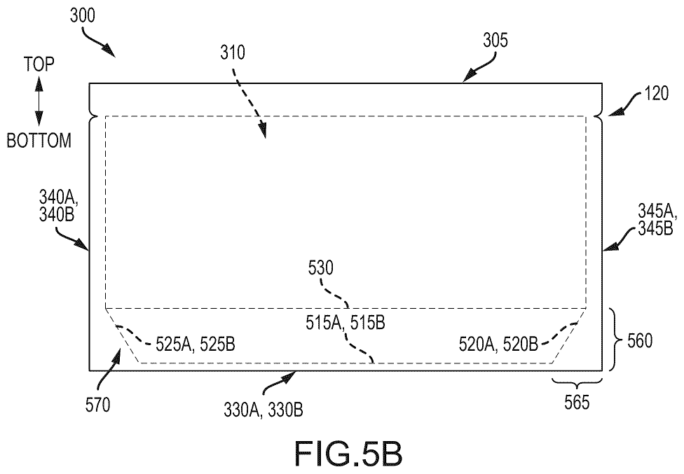

Package 305 of system 300 may have a rectangular shape as in package 205 of system 200. However, as compared to interior cavity 210 of package 205 of system 200, interior cavity 310 of package 305 of system 300 may have other than a rectangular shape when package 305 is lying flat in a sealed, unopened state. In the non-limiting example illustrated in FIG. 4A, interior cavity 310 of package 305 has a polygonal shape with greater than four sides (e.g., six sides). This may be due to the inclusion, by design, of a fold up/pop out structure 350 positioned proximate to the second side 330, as described below in greater detail with reference to FIGS. 5A and 5B. As compared to the packages (e.g., 105, 205) of systems (e.g., 100, 200) of the present technology as described above, presence of fold up/pop out structure 350 in system 300 may add only marginally to the thickness of package 305 in its sealed, lying flat, state.

FIGS. 5A and 5B provide pan views of example component parts of the system 300 shown in FIGS. 4A-4C, according to some embodiments of the present technology. FIG. 5A provides details relating to the fold up/pop out structure 350, and the description of FIG. 5A, below, makes references to various features described above with reference to FIGS. 4A-4C. In some embodiments, a one-piece fold up/pop out structure 350 may have two opposing long sides that are parallel to one another, and which define edges 515A and 515B having equal lengths. Midway between edges 515A and 515B, fold up/pop out structure 350 may include a centerline, denoted by the dashed line labeled 530 in FIG. 5A. In the example shown in FIGS. 5A and 5B, centerline 530 may have a length that is greater than the lengths of the long sides defining edges 515A and 515B of fold up/pop out structure 350. Fold up/pop out structure 350 may include four short sides defining two pairs of edges: a first pair of edges 525A and 525B, and a second paid of edges 520A and 520B. In some embodiments, the aforementioned four short sides defining edges 520A, 520B, 525A and 525B may have the same lengths, where those lengths are shorter than both the lengths of centerline 530 and of the two long sides defining edges 515A and 515B.

As shown in the bottom panel of FIG. 5A, for example and without limitation, centerline 530 may further define a fold line, whereby the fold up/pop out structure 350 formed of one piece may be folded in half to thereby assume a trapezoidal shape, as also shown by the structure in dashed lines also labeled 350 in FIG. 4A. In other examples, fold up/pop out structure 350 may be formed of two pieces that may be adhered, sealed, fused, or otherwise coupled to each other at their longest edges (e.g., with centerline 530 defining longest sides of two trapezoidal shaped pieces of fold up/pop out structure 350). In one embodiment, a material of construction of fold up/pop out structure 350 may be the same as a material construction of package 305.

The edges (515A, 515B, 520A, 520B, 525A, 525B), as well as centerline 530, of fold up/pop out structure 350 are also depicted as dotted lines in the top panel of FIG. 5A. The top panel of FIG. 5A also depicts package 305 unfolded, with the first side 325 of package 305 defining a fold line. Features labeled 305A and 305B are opposing, planar, surfaces of package 305 that face the exterior environment with package 305 in its folded over, sealed and unopened, state. Features labeled 535A and 535B are the corresponding opposing, planar, surfaces of package 305 that face the interior cavity 310 with package 305 in its folded over, sealed and unopened, state.

Still referring to the top panel of FIG. 5A, in the aforementioned example of a two piece fold up/pop out structure 350, longest (e.g., centerline) side 530 of each piece may be adhered, sealed, fused, or otherwise coupled to respective second sides (330A, 330B) of package 305 in its unfolded state. Next, in a non-limiting example method or process of forming system 300, package 305 may be folded over at its first side 325 fold line and, concurrently and/or after that folding step, the three other sides of fold up/pop out structure 350 may be adhered, sealed, fused, or otherwise coupled together. That is, in the example, edge 515A may be coupled to edge 515B, edge 520A may be coupled to edge 520B, and edge 525A may be coupled to edge 525B. This may yield the trapezoidal shaped fold up/pop out structure 350 as shown by the structure in dashed lines labeled 350 in FIG. 4A for package 305 lying flat in its sealed, unopened state, as for storage before opening and use.

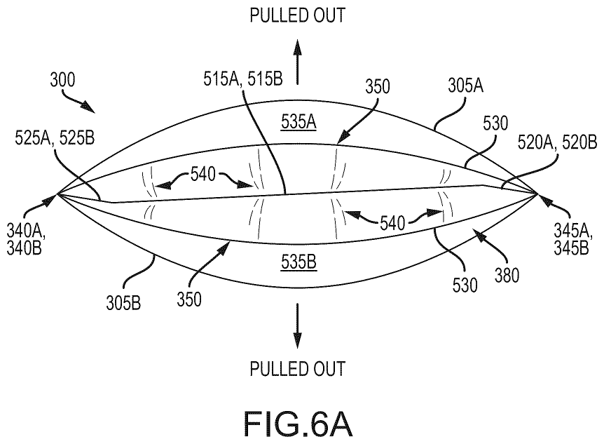

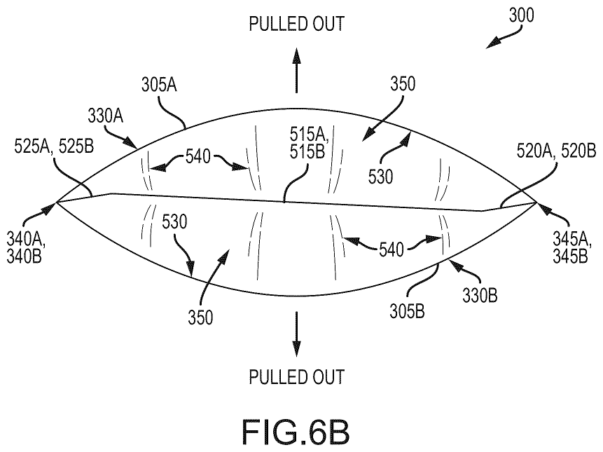

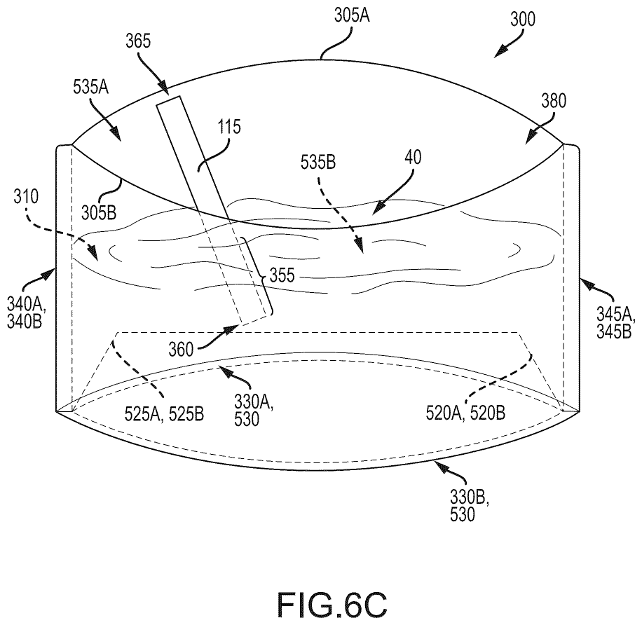

In the example described with reference to the top panel of FIG. 5A, respective second sides (330A and 330B) having sides 530 (e.g., that define the centerline) of fold up/pop out structure 350 coupled thereto may not be adhered, sealed, fused, or otherwise coupled to one another for reasons described in detail below with reference to FIGS. 6A-6C. Finally, in this example, respective short sides (340, 345) of package 305 in its folded state may be adhered, sealed, fused, or otherwise coupled to one another to thereby provide the sealed package 305 in its final form for use and with urine sample test strip 115 contained in its interior cavity 310. That is, in the example, first short side 340A may be coupled to the other first short side 340B, and second short side 345A may be coupled to the other second short side 345B. In doing so, two bottom corners are defined by junctures of coupled edges 520A and 520B, and coupled edges 525A and 525B, with coupled short sides 345A and 345B, and coupled edges 340A and 340B, respectively, may be sealed so as to ensure that no air, of liquid, flow communication is possible between interior cavity 310 and the exterior environment of package 305. In the aforementioned final form, the fold up/pop out structure 350 may be positioned proximate to a bottom of package 305, again as shown in FIG. 4A.

In another example, package 305 may be assembled, or otherwise formed, using the one-piece fold up/pop out structure 350 (e.g., as shown in the bottom panel of FIG. 5A). As compared to the example described above with respect to the top panel of FIG. 5A for a two-piece fold up/pop out structure 350, one piece fold up/pop out structure 350 may be folded along centerline 530. Next, package 305 may be folded over its first side 325. The longest side 530 (e.g., that defines the centerline) of fold up/pop out structure 350 may be inserted upward and in between interior surfaces 535A and 535B of the folded package 305. Edge 515A may be adhered, sealed, fused, or otherwise coupled to package 305 bottom, second side 330A, and edge 515B may be adhered, sealed, fused, or otherwise coupled to package 305 bottom, second side 330B. Edges 520A and 520B may be adhered, sealed, fused, or otherwise coupled together. Likewise, edge 525A may then be adhered, sealed, fused, or otherwise coupled to edge 525B.

Finally, the two sets of short sides may be adhered, sealed, fused, or otherwise coupled together. That is, short side 340A may be coupled to short side 340B, and short side 345A may be coupled to short side 345B. A juncture of one end of long side 330 and edges 525A and 525B with coupled short sides 340A and 340B may be sealed so as to ensure that no air, of liquid, flow communication is possible between interior cavity 310 and the exterior environment of package 305. Likewise, a juncture of the other end of long side 330 and edges 520A and 520B with coupled short sides 345A and 345B may be sealed for the same reasons. With centerline side 530 not coupled to either interior surface (535A, 535B) except at its opposite end points, it floats and may move and/or be manipulated independently of the second sides (330A, 330B) of package 305. In the final form of package 305 in this second example, the fold up/pop out structure 350 may be positioned proximate to a bottom of package 305. The configuration of system 300 after being formed in the manner of the presently discussed example using the one-piece fold up/pop out structure 350 is shown in FIG. 5B. The side views of the embodiment of system 300 shown in FIG. 5B are substantially the same as shown in FIGS. 4B and 4C. In the non-limiting example shown in FIG. 5B, rather than the respective short sides (340A/B, 345A/B) being coupled together along their entire lengths, a portion of those lengths proximate to package 305 bottom, second sides 330A/B may be left uncoupled thereby providing slits 560. In combination with portions 565 of package 305 bottom, second sides 330A/B proximate to the bottom corners of package 305, the slits 560 provide four flaps 570 that may also expand outwardly with the expansion of the fold up/pop out structure 350 along with expansion of opening 380 in operation of system 300. In this way, flaps 570 being expanded may facilitate not only maintenance of opening 380 in an expanded state, but also stable placement of the bottom of the package 305 to enable the package 305 in the opened state and containing urine 40 or other liquid to stand upright on a surface. This aspect of the present technology is further described below with reference to FIG. 9D.

FIG. 6A is a top view of system 300 in an open top and expanded state, and assembled or otherwise formed of the component parts shown in FIG. 5A, according to still other embodiments of the present technology. FIG. 6B is a bottom view of system 300 shown in FIG. 6A. FIG. 6C is a perspective view of system 300 shown in FIGS. 6A and 6B and also depicts a use case thereof. With further reference being made to FIGS. 4A-4C and 5, upon package 305 of system 300 being opened by way of, for example, means 120, the user may pull out opposing first sides 325A and 325A, as indicated by the arrows labeled “Pulled Out” in FIGS. 6A and 6B. Alternatively, or additionally, the user may push together opposing short sides 340 and 345. In either or both of those cases, an area or extent of an opening 380 of the opened package 305 of system 300 may be expanded, and a concurrent expansion of fold up/pop out structure 350 may advantageously facilitate maintenance of the opening 380 in its expanded state. In that state, portions(s) of interior surface(s) (535A and/or 535B) and/or fold up/pop out structure 350 may have crinkling, folding, deformation, etc. (labeled 540 in FIGS. 6A and 6B). Yet, given that the package 305 of system 300 is sealed, this will not have any functional impact during use of system 300 for purpose of containing urine 40 of a subject under test.

In an example use case, as shown in FIG. 6C, a user performing a test of a sample of urine 40 may use one or two of his or her hands to squeeze sides 340 and 345 of unsealed, opened package 305 together to create and further widen the opening 380 of the opened package 305. The test subject may then urinate the sample of urine 40 into interior cavity 310 through opening 380 while holding sides 340 and 345 of package 305. In an example of this use case according to the present technology, the user may next read his or her result directly from the functional area 355 proximate second end 360 opposite the first end 365 of test strip 115, with at least the functional area 355 submersed in the urine 40 sample for an amount of time for a particular test to be effectively and accurately read (e.g., per instructions provided with system 300 for a particular test). In another example, the user (e.g., patient) providing the sample of urine 40 may display the submersed test strip 115 to another person (e.g., medical practitioner) to read the result displayed on their test strip 115 functional area 355. Alternatively, the person (patient) may just present their urine 40 sample to the medical practitioner for the practitioner to submerge the test strip and perform the test. Means 765 for resealing or reclosing the opened package 305 may facilitate mess-free transport from the liquid 40 sampling location to the testing location, however far apart they may be. Such means 765 may also be useful where a liquid 40 sample may need to be placed on ice in a cooler. Having the ability to reseal or reclose package 305 may thus prevent infiltration of material other than the liquid 40 sample under test into package 305 during transport.

Still referring to FIG. 6C, in another example use case, the urine sample test subject may be a mammal other than a human being. In such cases, the user of system 300 may conveniently position the opening 380 of the unsealed, opened package 305 to collect the urine 40 sample from the non-human mammal test subject, and then proceed according to the above-described process for determining the test result for analyte(s) of interest. Accordingly, system 300 may find advantageous uses in both human and veterinary diagnostic applications in clinical settings, at home, or in the field. It can be appreciated from study of the present disclosure that system 300 (and likewise system 100 and/or 200) can be used to test for analytes on any liquid sample of interest. System 300 (and likewise system 100 and/or 200) is not limited just to urine sample testing of human or animal subjects, but rather can be advantageously utilized for collection and containment of liquids generally to be tested according to the present technology. One such non-limiting example may be testing of pH or chlorination level of water samples from a swimming pool or hot tub.

In any of the above-described use case examples according to FIG. 6C, thermometer(s) 190, when present, may provide to the user of system 300 a visual indication of the temperature of the urine 40 sample contained in the interior cavity 310 of package 305. This may be particularly useful in a clinical setting in order to detect attempted subterfuge, as in the context of urine sample testing for prohibited drug substances present in a human or animal subject. Should a sample of urine 40 of such a subject be indicated by thermometer(s) 190 to be outside an expected range of temperatures given a particular period of time elapsing since collection of the urine 40 sample, the sample may be flagged for further investigation (e.g., resampling of the subject, etc.).

In yet other embodiments, the package 305 of system 300 according to the present technology as described herein may be of a trapezoidal shape, it may be square shaped, or it may be triangular shaped (e.g., equilateral, isosceles, or scalene), without departing from the description of system 300 provided above according to the present technology and with reference to FIGS. 4A-4C, 5, and 6A-6C. In such examples, not shown in the figures, the fold up/pop out structure 350 and its manner of fitting and/or coupling to package 305 may be adjusted accordingly to provide the same, or analogous, advantageous ends as described above with respect to FIGS. 4A-4C, 5, and 6A-6C, again without departing from the scope and spirit of the present disclosure.

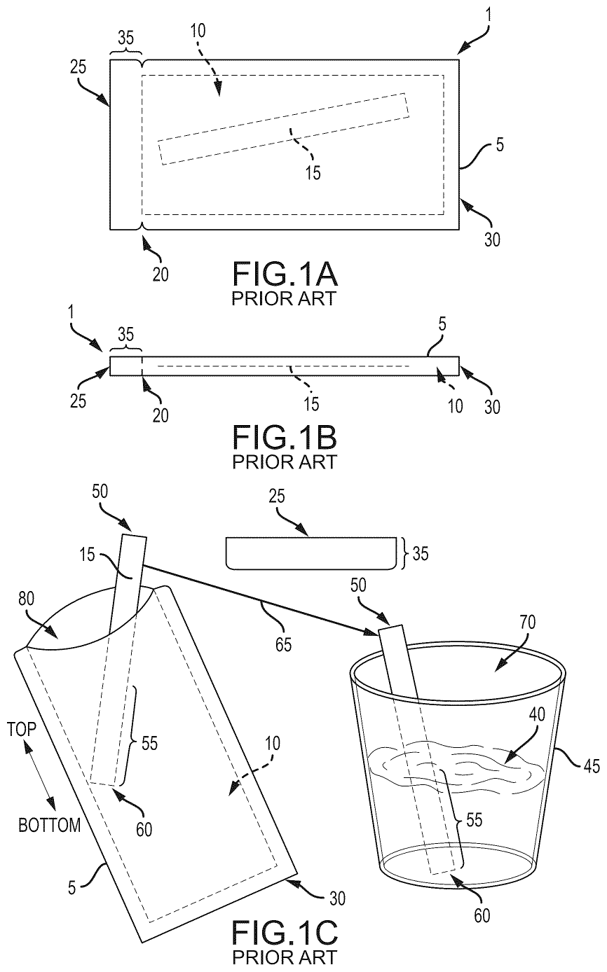

As compared to the known system (e.g., system 1) and associated method(s) of use as described above with reference to FIGS. 1A-1C, the fold up/pop out structure 350 of system 300 positioned proximate to the bottom of the package 305 and coupled to the at least a portion of interior surfaces (535A, 535B) of the interior cavity 310 facilitates, with the bottom of the package 305 in an expanded state, not only maintenance of opening 380 in an expanded state, but also stable placement of the bottom of the package 305 to enable the package 305 in the opened state to stand upright on a surface. Furthermore, users of the systems according to the present technology may dispense with use of cups 45. This reduces both the storage space required and volume of waste products generated, and enables the disclosed systems to be carried and used anywhere and at any convenient time. This is particularly advantageous for users of the systems and associated methods as disclosed herein because in some use cases (e.g., urine sample testing for ovulation status) the user and test subject may wish to test their urine 40 frequently and multiple times per day (e.g., including while they are in a workplace setting, outdoors, or on vacation, in addition to their home setting).

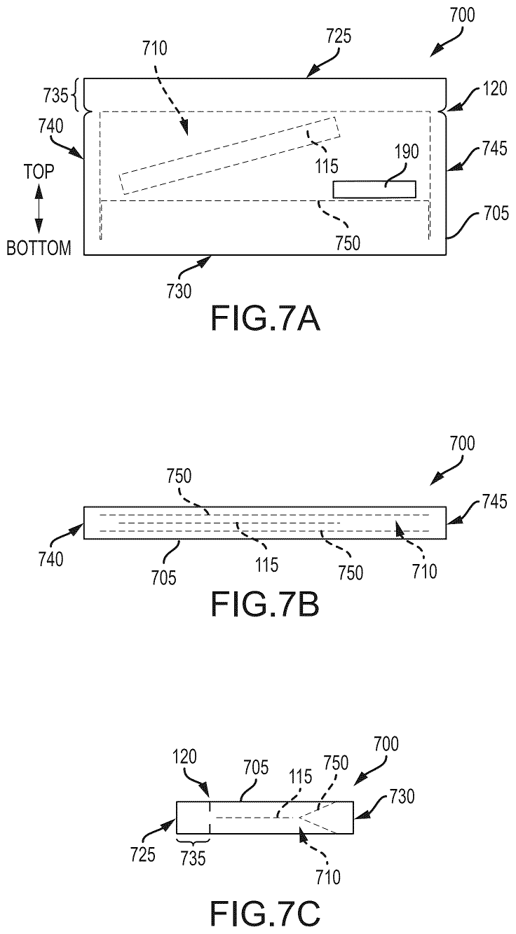

FIG. 7A is a plan view of a system 700 for performing a urine sample test, according to still another embodiment of the present technology. FIG. 7B is a side view of a long side of the system 700 shown in FIG. 7A. FIG. 7C is a side view of a short side of the system 700 shown in FIG. 7A. In an analogous manner as described above with reference to FIGS. 4A-4C, system 700 includes package 705, interior cavity 710, urine sample test strip 115, means 120 (e.g., cuts, notches, perforations, or the like) for user to open (e.g., unseal) the otherwise sealed package 705, corner sections 735, first 725 and second sides 730, short sides (740, 745), and thermometer(s) 190.

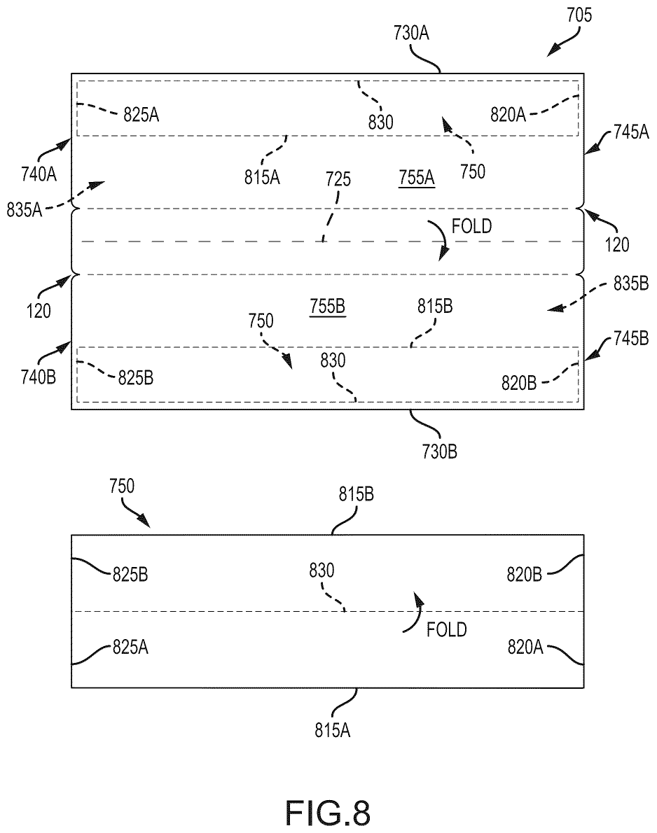

Package 705 of system 700 may have a rectangular shape as in package 305 of system 300. However, as compared to interior cavity 310 of package 305 of system 300, interior cavity 710 of package 705 of system 700 may have a rectangular shape when package 705 is lying flat in a sealed, unopened state. In the non-limiting example illustrated in FIG. 7A, interior cavity 710 of package 705 has four sides (e.g., rectangular), where two opposing short interior sides of interior cavity 710 have lengths that are less than short sides 740 and 745 of package 705 overall. This may be due to the inclusion, by design, of a fold up/pop out structure 750 positioned proximate to the second side 730, as described below in greater detail with reference to FIG. 8. As compared to the packages (e.g., 105, 205) of systems (e.g., 100, 200) of the present technology as described above, presence of fold up/pop out structure 750 in system 700 may add only marginally to the thickness of package 705 in its sealed, lying flat, state.

FIG. 8 provides pan views of example component parts of the system 700 shown in FIGS. 7A-7C, according to some embodiments of the present technology. FIG. 8 provides details relating to the fold up/pop out structure 750, and the description of FIG. 8, below, makes references to various features described above with reference to FIGS. 7A-7C. In some embodiments, a one-piece fold up/pop out structure 750 may have two opposing long sides that are parallel to one another and which define edges 815A and 815B having equal lengths. Midway between edges 815A and 815B, fold up/pop out structure 750 may include a centerline, denoted by the dashed line labeled 830 in FIG. 8. In the example shown in FIG. 8, centerline 830 may have a length that is equal to the lengths of the long sides defining edges 815A and 815B of fold up/pop out structure 750. Fold up/pop out structure 750 may include four short sides defining two pairs of edges: a first pair of edges 825A and 825B, and a second pair of edges 820A and 820B. In some embodiments, the aforementioned four short sides defining edges 820A, 820B, 825A and 825B may have the same lengths, where those lengths are shorter than both the lengths of centerline 830 and of the two long sides defining edges 815A and 815B.

As shown in the bottom panel of FIG. 8, for example and without limitation, centerline 830 may further define a fold line, whereby the fold up/pop out structure 750 formed of one piece may be folded in half to thereby assume a rectangular shape as shown by the structure in dashed lines also labeled 750 in FIG. 7A. In other examples, fold up/pop out structure 750 may be formed of two pieces that may be adhered, sealed, fused, or otherwise coupled to each other at their longest edges (e.g., with centerline 830 defining sides of two rectangular shaped pieces of fold up/pop out structure 750). In one embodiment, a material of construction of fold up/pop out structure 750 may be the same as a material construction of package 705.

The edges (815A, 815B, 820A, 820B, 825A, 825B), as well as centerline 830, are also depicted as dotted lines in the top panel of FIG. 8. The top panel of FIG. 8 also depicts package 705 unfolded, with the first side 725 of package 705 defining a fold line. Features labeled 755A and 755B are opposing, planar, surfaces of package 705 that face the exterior environment with package 705 in its folded over, sealed and unopened, state. Features labeled 835A and 835B are the corresponding opposing, planar, surfaces of package 705 that face the interior cavity 710 with package 705 in its folded over, sealed and unopened, state.

Still referring to the top panel of FIG. 8, in the aforementioned example of a two-piece fold up/pop out structure 750, the side 830 (e.g., that defines the centerline) of each piece may be adhered, sealed, fused, or otherwise coupled to respective bottom, second sides (730A, 730B) of package 705 in its unfolded state. Next, in a non-limiting example method or process of forming system 700, package 705 may be folded over at its first side 725 fold line. Concurrently and/or after that folding step, the three other sides of fold up/pop out structure 750 may be adhered, sealed, fused, or otherwise coupled together. That is, in the example, edge 815A may be coupled to edge 815B, edge 820A may be coupled to edge 820B, and edge 825A may be coupled to edge 825B. However, in the illustrated embodiment, edges 820A and 820B are not coupled to sides 745A and 745B, and edges 825A and 825B are not coupled to sides 740A and 740B. The enables sides defined by edges 815A/B, 820A/B and 825A/B of pop out/fold up structure 750 to “float” within the interior cavity 710 of package 705. This may yield the rectangular shaped fold up/pop out structure 750 as shown by the structure in dashed lines labeled 750 in FIG. 7A for package 705 lying flat, as for storage before use.

In the example described with reference to the top panel of FIG. 8, respective second sides (730A and 730B) may not be adhered, sealed, fused, or otherwise coupled to one another for reasons described in detail below with reference to FIGS. 9A-9D. Finally, in this example, respective short sides (740, 745) of package 705 in its folded state may be adhered, sealed, fused, or otherwise coupled together to thereby provide the sealed package 705 in its final form for use and with urine sample test strip 115 contained in the interior cavity 710. That is, in the example, first short side 740A may be coupled to the other first short side 740B, and second short side 745A may be coupled to the other second short side 745B. In doing so, two bottom corners defined by junctures of coupled edges 820A and 820B, and coupled edges 825A and 825B, with coupled short sides 745A and 745B, and coupled sides 740A and 740B, respectively, may be sealed so as to ensure that no air, of liquid, flow communication is possible between interior cavity 710 and the exterior environment of package 705. In the aforementioned final form, the fold up/pop out structure 750 may be positioned proximate to a bottom of package 705, again as shown in FIG. 7A.

In another example, package 705 may be assembled, or otherwise formed, using the one-piece fold up/pop out structure 750 (e.g., as shown in the bottom panel of FIG. 8). As compared to the example described above with respect to the top panel of FIG. 8 for a two-piece fold up/pop out structure 750, one piece fold up/pop out structure 750 may be folded along centerline 830. Next, package 705 may be folded over its first side 725. The side 830 (e.g., the defines the centerline) of fold up/pop out structure 750 may be inserted upward and in between interior surfaces 835A and 835B of the folded package 705. Edge 815A may then be adhered, sealed, fused, or otherwise coupled to bottom, second side 730A of package 705. Edges 820A and 825A may then be adhered, sealed, fused, or otherwise coupled to sides 745A and 740A, respectively. Likewise, edge 815B may then be adhered, sealed, fused, or otherwise coupled to bottom, second side 730B of package 705, and edges 820B and 825B may then be adhered, sealed, fused, or otherwise coupled to sides 745A and 740A.

Finally, the two sets of short sides may be adhered, sealed, fused, or otherwise coupled together. That is, short side 740A may be coupled to short side 740B, and short side 745A may be coupled to short side 745B. In doing so, a seal between edge 820A coupled to the corresponding portion of short side 745A, edge 815A coupled to bottom, second side 730A, and edge 825A coupled to the corresponding portion of short side 740A, along with a seal between edge 820B coupled to the corresponding portion of short side 745B, edge 815B coupled to bottom, second side 730B, and edge 825B coupled to the corresponding portion of short side 740B, may be established so as to ensure that no air, of liquid, flow communication is possible between interior cavity 710 and the exterior environment of package 705. With centerline 830 not coupled to either interior surface (835A, 835B) except perhaps at its opposite end points, it “floats” and may move and/or be manipulated independently of the opposing interior surfaces (835A, 835B) of package 705. In the final form of package 705 in this second example, the fold up/pop out structure 750 may be positioned proximate to a bottom of package 705, again as shown in FIG. 7A. The configuration of system 700 after being formed in the manner of the presently discussed example using the one-piece fold up/pop out structure 750 results in substantially the same configuration discussed below with respect to FIGS. 9A-9D (swapping positions of the features labeled 830 and 815A/B). The side views of the embodiment of system 700 shown in FIGS. 9A-9D are substantially the same as shown in FIGS. 7B and 7C. In the non-limiting example shown in FIG. 5B, rather than the respective short sides (740A/B, 745A/B) being coupled together along their entire lengths, a portion of those lengths proximate to package 705 bottom, second sides 730A/B may be left uncoupled thereby providing slits 770. The slits 770 provide four flaps 775 that may also expand outwardly with the expansion of the fold up/pop out structure 750 along with expansion of opening 780 in operation of system 700. In this way, flaps 775 being expanded may facilitate not only maintenance of opening 780 in an expanded state, but also stable placement of the bottom of the package 705 to enable the package 705 in the opened state and containing urine 40 or other liquid to stand upright on a surface. This aspect of the present technology is further described below with reference to FIG. 5B.

FIG. 9A is a top view of the system 700 shown in FIGS. 7A-7C in an open top and expanded state, and assembled or otherwise formed of the component parts shown in FIG. 8, according to still other embodiments of the present technology. FIG. 9B is a bottom view of the system 700 shown in FIG. 9A. FIG. 9C is a side view of the system 700 shown in FIGS. 9A and 9B. FIG. 9D is a perspective view of the system 700 shown in FIGS. 9A-9C. With further reference being made to FIGS. 7A-7C and 8, upon package 705 of system 700 being opened by way of, for example, means 120, the user may pull out opposing first sides 725A and 725A, as indicated by the arrows labeled “Pulled Out” in FIGS. 9A-9C. Alternatively, or additionally, the user may push together opposing short sides 740 and 745. In either or both of those cases, an area or extent of an opening 780 of the opened package 705 of system 700 may be expanded, and a concurrent expansion of fold up/pop out structure 750 may advantageously facilitate maintenance of the opening 780 in its expanded state. In that state, portions(s) of interior surface(s) (835A and/or 835B) and/or fold up/pop out structure 750 may have crinkling, folding, deformation, etc. (labeled 840 in FIGS. 9A-9C). Yet, given that the package 705 of system 700 is sealed, this will not have any functional impact during use of system 700 for purpose of containing urine 40 of a subject under test.

In another non-limiting example use case, discussed with reference to FIG. 9D, a user performing a test of a sample of urine 40 may use one or two of his or her hands to squeeze sides 740 and 745 of unsealed, opened package 705 together to create and further widen the opening 780 of the opened package 705. The test subject may then urinate the sample of urine 40 into interior cavity 710 through opening 780 while holding sides 740 and 745 of package 705. Similar to the use case described above with reference to FIG. 4C, the user may next read his or her result directly from the test strip 115 according to the test instructions (e.g., after test strip 115 functional area 355 was submersed in the urine 40 sample for an amount of time for a particular test to be read after a prescribed amount of time has elapsed). In another example, the user (e.g., patient) providing the sample of urine 40 may display the submersed test strip 115 to another person (e.g., medical practitioner) to read the result displayed on their test strip 115 functional area 355. Alternatively, the person (patient) may just present their urine 40 sample to the medical practitioner for the practitioner to submerge the test strip and perform the test.

In the illustrated example shown in FIGS. 9A and 9D, a user of system 700 may, in addition to, or instead of, squeezing sides 740 and 745 together while urinating into interior cavity 710 of package 705 through the opening 780, hold a handle 790 coupled (e.g., as a hinge-like attachment) to a portion of at least one of the two short sides (740, 745) of package 705 and/or to a portion of at least one of the exterior surfaces (755A, 755B) of package 705. In an example, handle 790 is C- or U-shaped and may be coupled to short side 740 or short side 745 of package at the open portion of the C- or U-shape, as shown for example and without limitation in FIG. 9D for an unfurled handle 790 state. Referring now also to FIG. 9A, in an unfurled state (e.g., for system 700 lying flat in a packaged state as described herein), a portion of handle 790 (e.g., distal from the open portion of the C- or U-shaped handle 790) may be removably attached to a portion of one of the exterior surfaces (e.g., 755A) of package 705.

In some embodiments, the aforementioned removable attachment may be accomplished by way of a means 795 (e.g., a weak adhesive, a weak weld or joint, a band, and the like) for removably coupling a portion of handle 790 to a corresponding portion of the exterior surface of package 705. In the example shown in FIGS. 9A and 9D, means 795 may be a tacky adhesive material that is strong enough to hold the portion of handle 790 to exterior package surface 755A, yet not so strong to prevent system 700 user from easily peeling the portion of handle proximate the means 795 from surface 755A and unfurling or unfolding handle 790 for use. In the case of tack and/or adhesive type means 795, a residue may remain on the portion of handle 790 and/or on the portion of package 705 exterior surface, but without any detrimental functional impact on the operation or performance of system 700 according to the present technology. Notably, it is emphasized that handle 790 and structure(s) similar to it and with analogous function(s) may be utilized with any of the other systems and methods described herein (e.g., system(s) 100, 200, and/or 300) to similar advantageous ends (e.g., convenient way of holding package 705 during urinating sample of urine into interior cavity 710 and/or carrying package 705 having urine 40 sample place to place).

FIG. 9E is a side view of the system 700 having handle 790 as shown in FIG. 9D. FIG. 9D illustrates an example of the handle 790 in a removably attached state in which two ends 792 of the open portion of the C- or U-shaped handle 790 are fixedly secured to portions of package 705 (e.g., alongside an edge of short side 745 and/or adhered between material of construction of near the edges of sides 745A and 745B). At least a portion of the handle 790 distal the ends 792 thereof can be less securely attached to an exterior surface (e.g., 755A) of package by way of means 795.

Still referring to FIG. 9D, in another example use case, the urine sample test subject may be a mammal other than a human being. In such cases, the user of system 700 may conveniently position the opening 780 of the unsealed, opened package 705 to collect the urine 40 sample from the non-human mammal test subject, and then proceed according to the above-described process for determining the test result for analyte(s) of interest. Accordingly, system 700 may find advantageous uses in both human and veterinary diagnostic applications in clinical settings, at home, or in the field. It can be appreciated from study of the present disclosure that system 700 can be used to test for analytes on any liquid sample of interest. System 700 is not limited just to urine sample testing of human or animal subjects, but rather can be advantageously utilized for collection and containment of liquids generally to be tested according to the present technology. One such non-limiting example may be testing of pH or chlorination level of water samples from a swimming pool or hot tub.

In any of the above-described use case examples according to FIG. 9D, thermometer(s) 190, when present, may provide to the user of system 700 a visual indication of the temperature of the urine 40 sample contained in the interior cavity 710 of package 705. This may be particularly useful in a clinical setting in order to detect attempted subterfuge, as in the context of urine sample testing for prohibited drug substances present in a human or animal subject. Should a sample of urine 40 of such a subject be indicated by thermometer(s) 190 to be outside an expected range of temperatures given a particular period of time elapsing since collection of the urine 40 sample, the sample may be flagged for further investigation (e.g., resampling of the subject, etc.).

In still other embodiments, the package 705 of system 700 according to the present technology as described herein may be of a trapezoidal shape, it may be square shaped, or it may be triangular shaped (e.g., equilateral, isosceles, or scalene), without departing from the description of system 700 provided above according to the present technology and with reference to FIGS. 7A-7C, 8, and 9A-9D. In the case of a trapezoidal shaped package 705, the shortest side of package 705 may either be the topmost side (e.g., 725), or the bottom most side (e.g., 730). Making the longest side be the bottom most side 730 of a trapezoidal shaped package 705 for system 700 may enable package 705 filled with urine 40 or other liquid to stand upright more stably on a surface as compared to a trapezoidal package 705 having it bottom side 730 shorter than its top side 725 (the same consideration may apply to other trapezoidal shaped packages of the other system embodiments described herein according to the present technology. In such examples, not shown in the figures, the fold up/pop out structure 750 and its manner of fitting and/or coupling to package 705 may be adjusted accordingly to provide the same, or analogous, advantageous ends as described above with respect to FIGS. 7A-7C, 8, and 9A-9D, again without departing from the scope and spirit of the present disclosure.

As compared to the known system (e.g., system 1) and associated method(s) of use as described above with reference to FIGS. 1A-1C, the fold up/pop out structure 750 of system 700 positioned proximate to the bottom of the package 705 and coupled to the at least a portion of interior surfaces (835A, 835B) of the interior cavity 710 facilitates, with the bottom of the package 705 in an expanded state, not only maintenance of opening 780 in an expanded state, but also stable placement of the bottom of the package 705 to enable the package 705 in the opened state to stand upright on a surface. Furthermore, users of the systems according to the present technology may dispense with use of cups 45. This reduces both the storage space required and volume of waste products generated, and enables the disclosed systems to be carried and used anywhere and at any convenient time. This is particularly advantageous for users of the systems and associated methods as disclosed herein because in some use cases (e.g., urine sample testing for ovulation status) the user and test subject may wish to test their urine 40 frequently and multiple times per day (e.g., including while they are in a workplace setting, outdoors, or on vacation, in addition to their home setting).

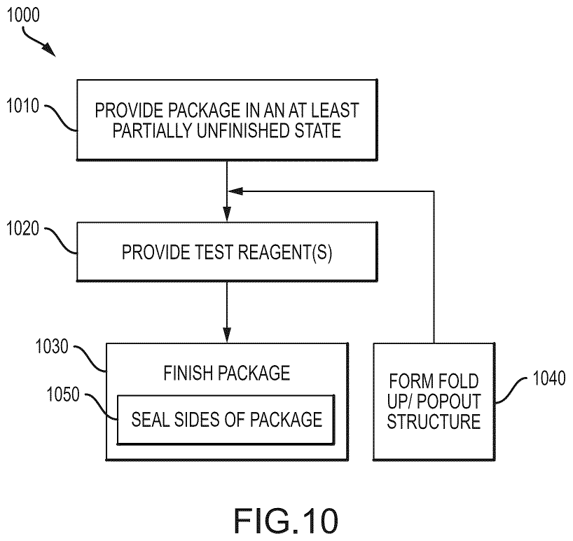

FIG. 10 is a flow chart of a method 1000 of manufacturing a test system for testing liquid samples according to some embodiments of the present technology. Method 1000 may be applied to, for example and without limitation, manufacturing any of the embodiments described above. Method 1000 may include the step of providing 1010 a package for the test system in an at least partially unfinished state. Method 1000 may include the step of providing 1020 at least one test reagent for the test system. Method 1000 may include the step of finishing 1030 the package with the at least one test reagent positioned in an interior cavity of the package in a finished state.

In some embodiments, method 1000 may include the step of forming at least one means for easing or otherwise facilitating the opening of one side of the package in its finished state. In an example, forming the at least one means for easing or otherwise facilitating the opening may be implemented, at least in part, in the method while the package is in the at least partially unfinished state. In one embodiment, method 1000 may include the step of forming means for alternately sealing and unsealing the package in an opened state during use. In an example, forming the means for alternately sealing and unsealing may be implemented, at least in part, in method 1000 while the package is in the at least partially unfinished state.

In another embodiment, method 1000 may include forming 1040 a fold up/pop out structure positioned proximate to a bottom of the package and coupled to at least a portion of interior surfaces of the interior cavity to thereby enable the bottom of the package to be expanded outward. In an example, forming the fold up/pop out structure may be implemented, at least in part, in method 1000 while the package is in the at least partially unfinished state.

In yet another embodiment, method 1000 may include the step of forming a handle configured to extend at or near normal from a side of the package during use thereof. In an example, forming the handle may be implemented, at least in part, in method 1000 while the package is in the at least partially unfinished state. In an example, forming the handle may be implemented, at least in part, in method 1000 while the package is in the finished state.

In still another embodiment, method 1000 may include the step of positioning a thermometer sufficiently with respect to the interior cavity of the package to enable a user to visually determine a temperature of the liquid sample contained in the package in the opened state. In an example, positioning the thermometer may be implemented, at least in part, in method 1000 while the package is in the at least partially unfinished state. In an example, positioning the thermometer may be implemented, at least in part, in method 1000 while the package is in the finished state.

In some embodiments, the providing step 1010 of method 1000 may include providing the package of the test system with a rectangle, square, trapezoid, or triangle shape. In another embodiment, the finishing step 1030 of the method may include the step of sealing 1050 all sides of the package in the at least partially unfinished state to thereby place the package in the finished state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize. For example, while features or steps are presented in a given order, alternative embodiments may have functionally equivalent features and/or steps, but in varying combinations or orders; likewise, some features and/or steps may be deleted, moved, added, subdivided, combined, and/or modified to provide alternative or subcombinations. Each of these features and/or steps may be implemented in a variety of different ways.

The teachings of the disclosure provided herein can be applied to other systems, not necessarily the system described above. The elements and acts of the various embodiments described above can be combined to provide further embodiments.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112(f), other aspects may likewise be embodied as a means-plus-function claim, or in other forms, such as being embodied in a computer-readable medium. (Any claims intended to be treated under 35 U.S.C. § 112(f) will begin with the words “means for”.) Accordingly, the applicant reserves the right to add additional claims after filing the application to pursue such additional claim forms for other aspects of the disclosure.

The detailed description provided herein may be applied to other systems, not necessarily only the system described above. The elements and acts of the various examples described above can be combined to provide further implementations of the invention. Some alternative implementations of the invention may include not only additional elements to those implementations noted above, but also may include fewer elements. These and other changes can be made to the invention in light of the above Detailed Description. While the above description defines certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention.

What is claimed is:

1. A test system for liquid samples, comprising:
a test strip for testing a liquid sample,
a flat and planar package, initially formed in a sealed or closed state, and defining an interior cavity to contain the test strip therein, wherein the package has at least three sides and is configured to be opened on a top side of the at least three sides of the package to expose the interior cavity to thereby enable access to the test strip by a user of the test system, wherein the package in an opened state has an opening at its top, and wherein the package comprises a fold up/pop out structure positioned proximate to a bottom of the package.

2. The test system of claim 1, wherein the top side has a length that is greater than at least one other side of the at least three sides of the package.

3. The test system of claim 1, wherein the fold up/pop out structure is coupled to at least a portion of interior surfaces of the interior cavity of the package to thereby enable the bottom of the package to be expanded outward from a flat and planar state.

4. The test system of claim 3, wherein the fold up/pop out structure positioned proximate to the bottom of the package and coupled to the at least a portion of interior surfaces of the interior cavity further enables, with the bottom of the package in an expanded state, maintenance of the package in the opened state with the opening at the top of the package remaining open.

5. The test system of claim 4, wherein maintenance of the package in the opened state with the opening at the top of the package remaining open further enables dispensing or placement by the user of the liquid sample into the exposed interior cavity through the opening at the top of the package.

6. The test system of claim 4, wherein the liquid sample is urine, and wherein maintenance of the package in the opened state with the opening at the top of the package remaining open further enables urination by the user into the exposed interior cavity through the opening at the top of the package.

7. The test system of claim 3, wherein the fold up/pop out structure positioned proximate to the bottom of the package and coupled to the at least a portion of interior surfaces of the interior cavity further enables, with the bottom of the package in an expanded state, stable placement of the bottom of the package on a surface to thereby enable the package in the opened state to stand upright on the surface.

8. The test system of claim 1, wherein the flat and planar package is rectangle shaped having two long sides and two short sides, and wherein a top side of the package is a first side of the two long sides.

9. The test system of claim 1, wherein the package is trapezoid shaped having at least a first side and a second, shorter side parallel to, and opposite, the first side.

10. The test system of claim 1, wherein the package is square shaped, and wherein a first side of the package is a top side thereof positioned opposite a second side of the package.

11. The test system of claim 1, wherein the package is triangle shaped, and wherein a first side of the package is a top side thereof.

12. The test system of claim 1, wherein the top side of the package includes means for opening the package via removal of at least part of the first side to thereby expose the interior cavity and create the opening at the top of the package.

13. The test system of claim 1, wherein the test strip is capable of qualitatively or quantitatively determining at least one of:
a pregnancy status;
an ovulation status; and
a presence or level of at least one of:
one or more drugs;
one or more drug metabolites;
one or more proteins;
one or more biomarkers;
glucose; and
ketone.

14. The test system of claim 1, wherein the test strip is capable of testing for: a presence or level of chlorination in, or a pH of, the liquid sample.

15. The test system of claim 1, further comprising a thermometer positioned sufficiently with respect to the interior cavity of the package to enable a user to visually determine a temperature of the liquid sample contained in the package in the opened state.

16. The test system of claim 1, further comprising a handle.

17. The test system of claim 1, further comprising means for alternately sealing and unsealing the package in the opened state.

18. The test system of claim 1, further comprising a plurality of test strips contained within the interior cavity of the package in the sealed or closed state.

19. A liquid sample test system, comprising:
a package initially formed to be flat and planar, and in a sealed or closed state to thereby define an interior cavity of the package; and
at least one test reagent for testing a liquid sample to be contained within the interior cavity of the package, wherein the at least one test reagent is contained within the interior package in the sealed or closed state, and
wherein the package is capable of being opened by a user on a top side of the package to expose the interior cavity of the package to an exterior of the package to thereby enable:
access to the at least one test reagent through an opening of the package in an opened state; and
placement of the liquid sample into the package through the opening of the package.

20. A method of manufacturing a test system for liquid samples, the method comprising:
providing a package for the test system in an at least partially unfinished state;
forming a fold up/pop out structure positioned proximate to a bottom of the package;
providing at least one test reagent for the test system; and
finishing the package with the at least one test reagent positioned in an interior cavity of the package in a finished state.
